(19) United States
(12) Patent Application Publication
(10) Pub. No.: US 2008/0294064 A1
(43) Pub. Date: Nov. 27, 2008

(54) Lancing Element, Lancing System and a Method for Skin Detection

(76) Inventors: Irio Calasso, Arth (CH); Oliver Bruendler, Adligenswil (CH); Michael Glauser, Rotkreuz (CH); Hans-Peter Wahl, Schopfheim (DE); Stephan Korner, Cham (CH)

Correspondence Address: ROCHE DIAGNOSTICS OPERATIONS INC., 9115 Hague Road, Indianapolis, IN, 46250-0457, US

(21) Appl. No.: 12/118,146

(22) Filed: May 9, 2008

Related U.S. Application Data
(63) Continuation of application No. PCT/EP2006/010802, filed on Nov. 10, 2006.

(30) Foreign Application Priority Data
Nov. 10, 2005 (EP) 05024477.1

(51) Int. Cl. A61B 5/151; A61B 5/053

(52) U.S. Cl. 600/547; 600/583

(57) ABSTRACT

In the case of a disposable lancing element for collecting body fluid through the skin comprising a support and a lancing member which distally projects therefrom and can pierce the skin in a lancing movement, it is proposed that a pretensioning means which tightens the skin in the area of the puncture site is arranged on the support and has a contact member which impacts the skin laterally from the lancing member during the lancing movement.

### REFERENCE

This application is a continuation of PCT/EP2006/010802 filed Nov. 10, 2006 which is based on and claims priority to European Patent Application No. EP 05024477.1 filed Nov. 10, 2005, which are hereby incorporated by reference.

### FIELD

The disclosure concerns a lancing element for collecting body fluid through or over the skin in particular as a disposable part for blood sugar tests comprising a support and a lancing member which rigidly projects therefrom and can pierce the skin in a lancing movement. The disclosure additionally concerns a lancing system in which such a preferably disposable lancing element can be used and a method for skin detection when taking samples in this manner.

### BACKGROUND

A system for withdrawing blood by means of a lancing element having a support-bound collecting unit is known from WO 2005/096941 A1 in which a pressure ring supported on the instrument side exerts pressure oh a finger that is pressed against it in order to provide sufficient blood in the compressed finger area and subsequently reducing pressure to prevent blood from escaping. In order to avoid contamination with blood and ensure an adequate increase of the body inner pressure, the pressure ring must have a diameter matching the dimensions of the finger pad as a result of which the body part is arched in the lancing area. The impact of the needle during the lancing propulsion then makes an inward depression in the skin until finally a lancing channel is generated depending on the skin type and skin thickness of the test subject.

Based on this the disclosure further develops the known systems in the prior art and to optimize a generic arrangement and a method especially with regard to a defined lancing depth where one aim of the invention is also a simplified system design which has advantages with regard to hygiene.

### SUMMARY

The teachings are based on the idea of pressing in the skin in the area of interaction of the lancing member by means of a skin tightener as an integral component of the lancing element. Accordingly it is proposed according to the disclosure that a pretensioning means which tightens the skin in the area of the puncture site and which has a contact member that impacts the skin during the lancing movement and can move back during this process in the opposite direction to the lancing member, is attached to the support. The contact member provides further points of support on the skin thus improving the pressure distribution in the region of the puncture site. Hence the contact member reduces the skin penetration by the lancing member. The contact member produces a concave or convex bulge of the skin at the puncture site so that the skin penetration by the impacting lancing member is reduced and the puncture channel is immediately generated. The lancing member is released while the contact member resting on the skin makes a relative backwards movement and the skin tightening directly at the puncture site allows the effect of skin-specific variations on the lancing depth to be substantially eliminated. Furthermore, contamination of the device with body fluid can be reliably avoided by the integral arrangement of the contact member on the preferably disposable lancing element. Another important advantage is that the complexity of the system or device and thus also its overall size can be reduced when the skin tightening function is implemented not on the instrument side but rather on the lancing element.

In its initial state the contact member is advantageously arranged in front of the lancing member in the lancing direction and on the skin it is braced by the support against a backwards movement during the lancing movement. This ensures that the lancing member penetrates the pretensioned skin by means of a simple forwards movement. In principle it may also be sufficient for the contact member to be located essentially at the same level as the lancing member such that the skin is tightened at least during entry into the blood generating skin zone.

Another advantageous embodiment provides that the pretensioning means, has an elastically and/or plastically compressible coupling member or one that is held by frictional lock which connects the contact member with the support. This enables an additional forward movement of the lancing element while the contact member already rests against the skin. In this connection it is also advantageous for the production process when the coupling member is formed by a folding arm which can be bent or folded at least one bending point. An additional spring excursion can be provided by supporting the contact member via a spring element.

In order to substantially cancel the contact force during the return movement into a collecting position, it is advantageous when the contact member can be automatically locked in a reset position that is proximally set back relative to the lancing member by means of a locking mechanism and in particular a catch.

It is advantageous for the blood collection when the contact member can be moved together with the support when the support is retracted while reducing or removing the contact pressure on the skin. This reduces the displacement of body fluid by the contact member.

With regard to the production process and the function it is advantageous, when the pretensioning means is attached as an integral component and preferably as one piece to the support. It is particularly preferable when the support, the lancing member and the pretensioning means are formed uniformly from one material as a disposable part. This is also advantageous for hygienic reasons because skin contact only occurs with a sterilizable article that is used once.

A further improvement provides that the lancing element is formed from a flat substrate and is in particular etched and that the pretensioning means is arranged as a part of the substrate in the substrate plane or is bent out of this substrate. Flexures of the contact member can also be advantageously generated from a flat substrate in the production process as arched or folding elements by means of appropriate two-dimensional etching.

According to a further advantageous embodiment the contact member has a point shaped or linear edge contour that can be pressed against the skin laterally next to the lancing member. This should occur as close as possible to the puncture site without colliding with the lancing member. Accordingly it is advantageous when the lancing member can be inserted into the skin at a lateral distance of less than 3 mm and preferably of 1 to 2 mm from the contact member.

In order to adjust the lancing depth, it is advantageous when a stop which limits the lancing depth of the lancing member is attached to or molded onto the support as one piece and has a skin contact surface that impacts at a defined proximal distance to the lancing member during the lancing movement.

In order to adjust the lancing depth, it is advantageous when the length of the stop which projects from the base member can be varied in the lancing direction preferably by means of a bending deformation or adjustable stop positions.

The lancing element can be designed as a simple lancet while a preferably capillary-active collecting structure which extends into the region of the lancing member is advantageous for a simultaneous collection of body fluid.

The invention also concerns a lancing system for collecting body fluid through or over the skin comprising a lancing drive and a lancing element according to the invention that can be moved forwards and backwards by means of this drive in a lancing movement.

A particularly advantageous variant envisages a position detector for detecting the position of the skin, preferably of the tightened skin, during the lancing movement. This enables a very accurate determination of the lancing depth without an “idle path” of the lancing member leading to errors due to skin indentation. The lancing movement can comprise a probing movement with a return movement before penetration or only one single forward and backward movement.

In this connection it is advantageous when the position detector probes the position of the skin tightened by the pretensioning means using the contact member and/or the lancing member as a sensor and when the position detector detects a change in capacitance or conductivity or force when it probes the skin. It is obvious that the lancing member can undertake a detection function by simultaneously being a capacitance, conductivity or force sensor relative to the skin surface.

According to another advantageous embodiment the lancing element is connected to a distance measuring unit which preferably operates incrementally to detect the relative position of the contact member and lancing member.

For a defined lancing process which is thus as pain-free as possible, it is advantageous when the lancing drive has a device, for adjusting the lancing depth of the lancing member relative to a detected reference position of the skin and preferably of the tightened skin.

Another aspect of the invention is to provide a system for skin detection for taking samples of body fluid in which the position of the skin relative to a movement axis of a lancing element is detected by a position detector wherein the skin is locally tightened during the position detection by a pretensioning means of the lancing element. In this connection it is advantageous when the lancing element is moved into a retracted position from the skin after the position detection and then the lancing movement is executed by means of a lancing drive. For the skin detection, the pretensioning means can be formed by a lancing member or a separate contact member of the lancing element. The lancing movement of the lancing element is advantageously controlled according to the detected skin position by a lancing drive in order to set a defined lancing depth.

With regard to the process, the aforementioned object in the sense of a skin detection for taking samples of body fluid in which the position of the skin is detected relative to a movement axis of a lancing element, is achieved in that the skin is locally tightened/during the position detection by a pretensioning means of the lancing element.

In order to execute the lancing movement more dynamically, it is advantageous when the lancing element is moved into a starting position that is retracted from the skin after the position has been detected and before a lancing movement. The pretensioning means can comprise a lancing member or a separate contact member of the lancing element. In this connection it is also advantageous when the lancing movement of the lancing element is controlled according to the detected skin position in order to set a defined lancing depth.

### BRIEF DESCRIPTION OF THE DRAWINGS

The invention is elucidated in more detail in the following on the basis of the examples of embodiments shown schematically in the drawing:

FIG. 1 shows a blood sugar measuring instrument comprising a lancing element designed to tighten the skin at the lancing site in a schematically simplified diagram;

FIGS. 2, 3 and 4 show further embodiments of a lancing element with a skin tightener;

FIGS. 5a-5f show the lancing element according to FIG. 1 in various positions during the lancing movement;

FIG. 6 shows an embodiment of a lancing element with a lockable skin tightener in a side-view;

FIG. 7 shows a further embodiment of a locking mechanism in a sectional view;

FIGS. 8a and 8b show a skin sensor on a lancing element in a sectional view in the initial state and lancing state;

FIGS. 9a and 9b show an additional ring or finger holder in connection with the lancing element;

FIGS. 10 and 11 show a diagram of two different lancing methods each in the initial and end position; and,

FIGS. 12a, 12b and 13 show lancing elements with adjustable spacers for adjusting the lancing depth in an illustrative diagram.

### DETAILED DESCRIPTION

The lancing elements 10 shown in the drawing have a support or a base part 12 as a holder, a lancing member 14 projecting from the holder in the lancing direction (distal) that is formed as a point and a pretensioning means 16 as a skin tightener that can be placed on the skin next to the lancing member.

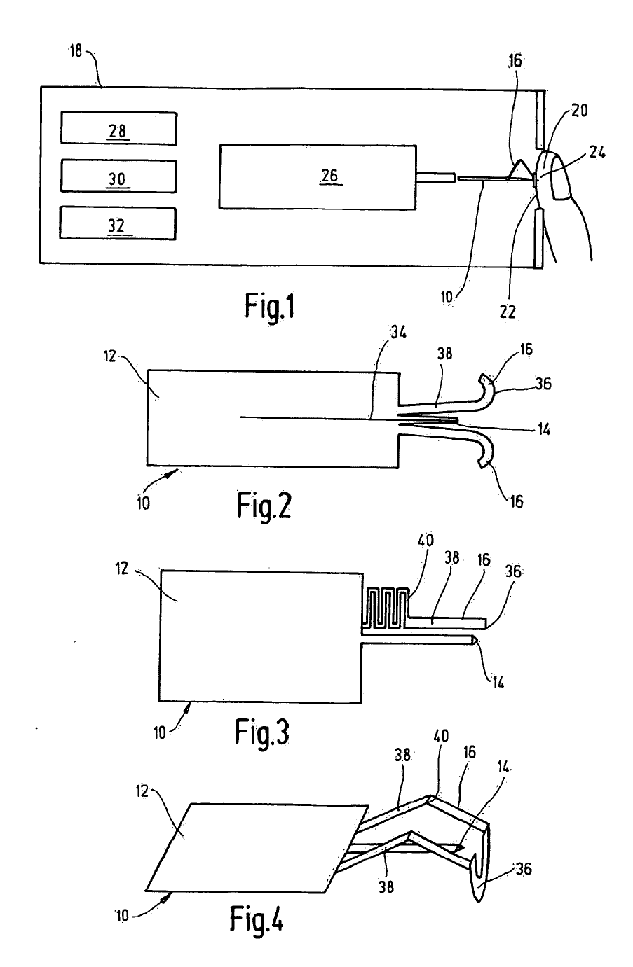

According to FIG. 1 such lancing elements 10 can be inserted into a handheld device 18 as single-use articles (so-called disposables) in order to collect body fluid i.e. blood and optionally also tissue fluid in a lancing movement especially for blood sugar self-tests. In this connection a body part and in particular the finger pad 20 is placed by the user on an opening in the device. Then a small amount of blood is collected at a puncture site by means of a lancing member 14 in a forward and backward lancing movement against the skin area 22 resting against the instrument opening and is preferably analyzed in the instrument 18.

For this purpose the instrument 18 has a lancing drive 26 which moves the inserted lancing element 10, a skin defector 28, a device that interacts therewith for adjusting the lancing depth 30 and further instrument component assemblies such as an analytical unit 32. The blood collected on the test element 10 can thus be utilized on-site for a blood sugar determination in an automated measuring process. Subsequently the used test element is disposed of and a new test element is provided preferably from an instrument magazine to ensure the most hygienic handling.

The test element 10 shown in FIG. 2 is etched uniformly from a flat substrate, for example stainless steel sheet, during which the lancing member 14 and the pretensioning means 16 are etched free together with the base part 12 as integral components lying in the plane of the substrate. In this manner it is possible to manufacture one-piece lancing elements formed from one part in a uniform process sequence that is suitable for mass production. In order to additionally integrate a collecting function, for the body fluid obtained at the puncture site, a capillary channel 34 that is semi-open on the longitudinal side runs across the base part 12 into the region of the lancing member 14.

The pretensioning means 16 is formed by a contact member 36 which, during the lancing movement, impacts the skin laterally before the lancing member 14 and forms a coupling part 38 which connects the contact member with the support 12. In this manner the pretensioning means 16 is moved together with the support 12 as an integral structure. The point shaped or linear edge contour of the contact member 36 is placed on the skin 22 during the forward movement of the lancing element before the lancing element 10 makes contact with the skin. During the further advance the distal length of the coupling part 38 is elastically and/or plastically deformed by the contact member 36 resting against the skin as it is moved back relative to the lancing member 14 such that the lancing element 14 punctures the previously tightened skin as elucidated in more detail in the following.

In the embodiment example shown in FIG. 3, the contact member 36 is also held at a slight lateral distance from the lancing member 14 and in front of the lancing member 14 in the lancing direction where a spring element 40 allows a limited return movement against the base member 12 during the lancing advance. In order to tighten the skin near to the puncture site, the lateral distance between the contact member 36 and the lancing member 14 should be less than 3 mm and preferably 1 to 2 mm in the active position.

Whereas in the embodiments according to FIGS. 2 and 3 the pretensioning means 16 is arranged in the substrate plane the embodiment example according to FIG. 4 envisages a double folding arm 38 as a coupling part which allows a distal shortening relative to the lancing member 14 at a bending point 40 that can be folded away from the substrate plane.

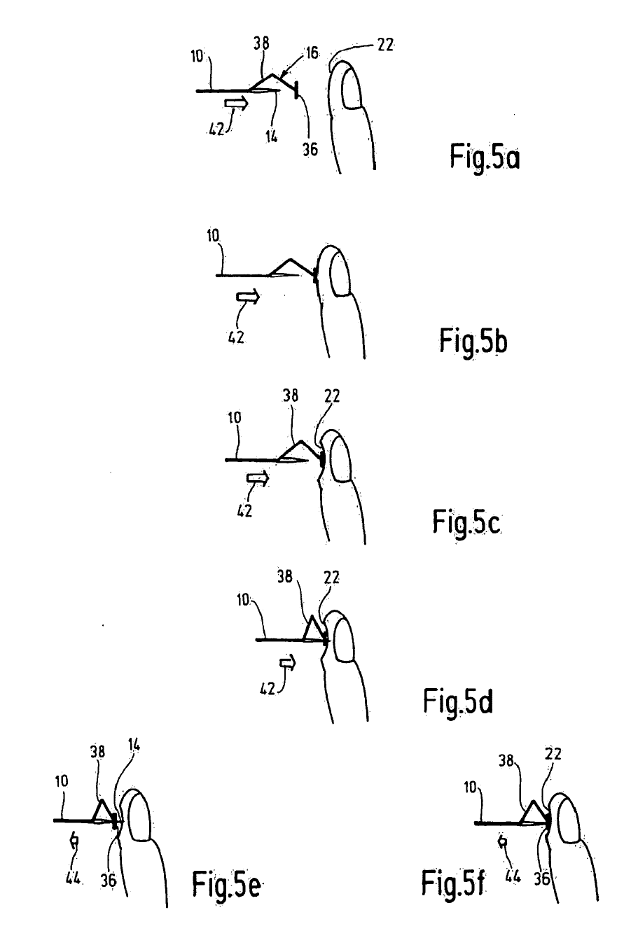

FIG. 5 illustrates the skin tightening during the lancing and collecting process in various stages of the movement. During the forward movement (arrow 42) the contact member 36 of the pretensioning means or of the skin tightener 16 is located in front of the lancing member (FIG. 5a) and thus impacts the skin 22 near to the intended puncture site (FIG. 5b). During the further advance the skin 22 is displaced with a defined force during which the folding arm 38 is compressed (FIG. 5c) until finally the needle is inserted into the pretensioned skin 22 (FIG. 5d).

After the intended puncture depth that is defined relative to the tightened skin has been reached, the lancing element is retracted in a return movement (arrow 44) to a collecting position that is punctured to a lesser depth. If the folding arm 38 was previously plastically deformed, or held back by a locking mechanism, the contact member 36 follows the return movement as shown in FIG. 5e. In this process the pressing force is substantially abolished and the skin 22 relaxes in the area of the puncture site. As a result less body fluid is displaced in the affected part of the skin and an adequate amount of fluid (microliter or less) can be collected in a short period. In the alternative shown in FIG. 5f the folding arm 38 was not plastically deformed during the insertion but only elastically pressed together by a certain length. Hence during the retraction the contact member 36 remains on the skin 22 while the return force of the folding arm 38 is reduced. Thus also in this case the displacement of the body fluid in the body part is less than at the maximum puncture depth.

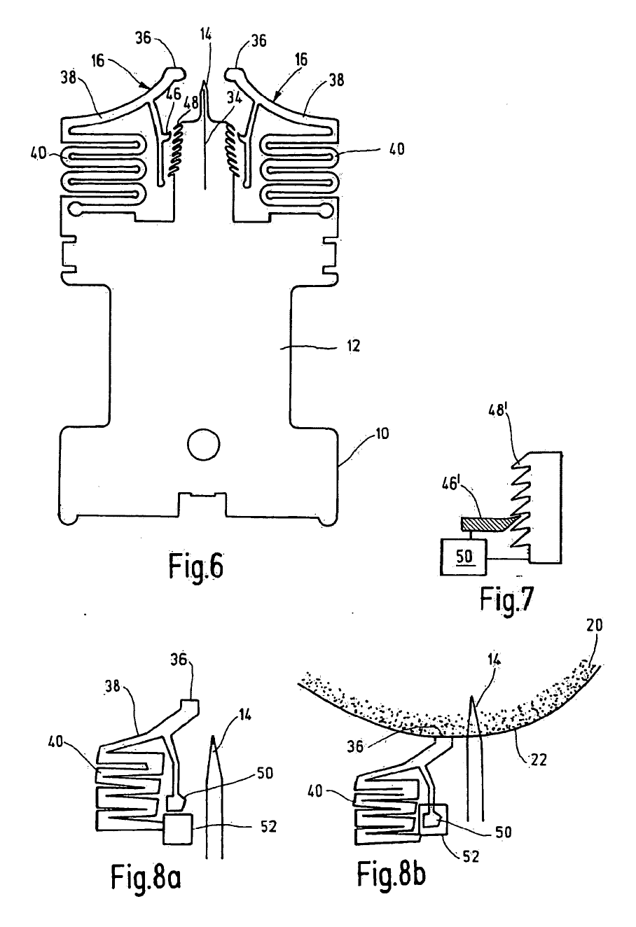

In the embodiments shown in the following figures, parts that have already been described above are provided with the same reference numerals. According to FIG. 6 a locking catch 46 is additionally molded on to the coupling part 38 which can be hooked into toothing 48 on the base member 12 to secure the reset position of the contact member 36. Thus a locking is achieved at the maximum advance of the lancing element 10 and corresponding elastic resetting of the pretensioning means 16 in contact with the skin 22 so that, similar to the embodiment according to FIG. 5e, the compressed pretensioner 16 no longer presses against the skin 22 during the return movement into the collecting position.

According to FIG. 7 it is also conceivable that a catch 46′ on the instrument engages in a locking manner in toothing 48′ of the pretensioning means 16 in a directionally dependent manner. This latch 46′ advantageously has a greater thickness than the toothing 48′ so that certain height tolerances of the relative positioning can be compensated. Suitable deflections of the toothing 48′ from the substrate plane are also conceivable for this purpose. Such an arrangement can basically also be used to register the number of latching clicks by means of a distance measuring unit 50 (for example by means of an electrical impulse counter) and in this maimer to determine the relative displacement between the contact member 36 and the lancing member 14 and to evaluate it in the context of a determination of the lancing depth.

FIG. 8 shows an embodiment in which the position of the tightened skin in the lancing direction is detected capacitatively by means of the skin detector 28 via the contact member 36. For this purpose an electrode arm 50 is arranged on the coupling part 38 which interacts with a fixed counter electrode 52 in the instrument as a displacement sensitive capacitor arrangement. As seen in FIG. 8, the maximum capacitance is achieved in the advance position with maximum skin tightening. This also allows a determination of the displacement relative to the lancing member 14 and thus its actual puncture depth for example by obtaining empirical comparative values by means of a calibration. The detection of the puncture depth can be even used during the forward movement as an input signal for the device for adjusting the puncture depth 30 which accordingly controls the lancing drive 26.

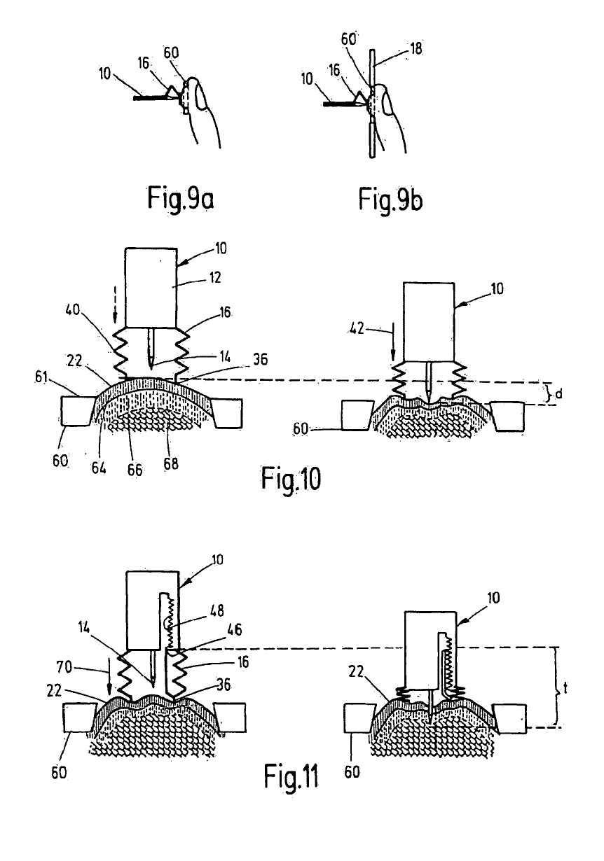

FIG. 9a shows the lancing element 10 in combination with a cone or pressure ring 60 as a finger receiver. Such a finger receiver 60 can according to FIG. 9b also be used in an opening of an instrument 18 shown FIG. 1. In any case the pretensioning means 16 acts within the ring boundary formed by the receiver 60 to prefix the skin 22.

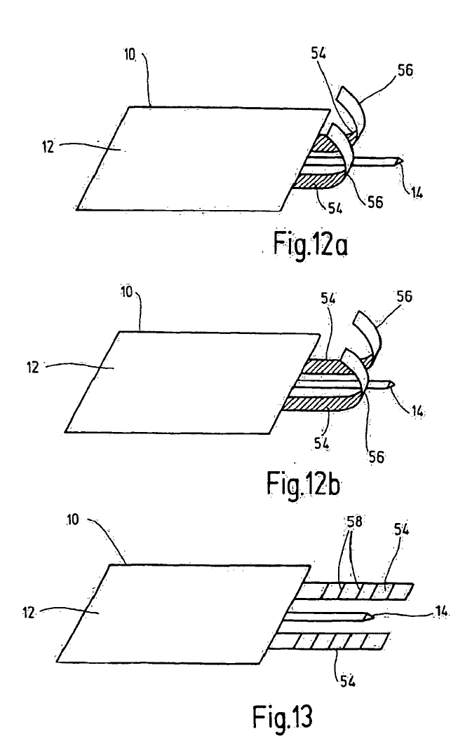

As shown in more detail in FIG. 10, the impact of the pretensioning means 16 on the skin 22 results in an improved pressure distribution during the lancing process. This is due to the fact that several points or lines of support are present optionally within an additional finger ring 60. As a result the maximum displacement d of the skin 22 before it is penetrated by the lancing member 14 is considerably less than without a pretensioning means. This applies to any skin type whether soft, medium or hard. In general it should be taken into consideration in this connection that the skin has a layered structure with the so-called stratum corneum 64 as the upper layer, the adjoining epidermis 66 and the underlying blood yielding zone 68.

In order to collect body fluid a skin contact can be firstly detected by the contact member 36 as shown in the left half of FIG. 10. In this case the contact member 36 is a reference for the skin surface but not necessarily a measuring sensor. Rather the sensory detection can take place according to FIG. 8 or by detecting a change of the required propulsion force on the drive side. In any case it is important that the lancing element 10 is manufactured sufficiently accurately in order to factor in the relative distance between the contact member 36 and lancing member 14. The lancing stroke is then executed on the basis of this initial position preferably using a distance or depth that is preset by the user at which only a small, substantially invariant skin displacement d occurs due to the advantageous effect of the pretensioning means 16.

It is also possible that a reference position on the instrument, for example the bearing surface 61 of the ring, is selected as the initial position for the lancing stroke, or it is possible to entirely do without a position detection when the maximum displacement d of the skin with the pretensioning means 16 is considerably less before the needle penetration than the variations of the skin bulge at the ring 60.

FIG. 11 shows a further lancing method with a force detector on the pretensioning means 16. In this process the distance traveled until a predetermined restoring force occurs is determined in a probing movement 70 for example on the basis of a first locking click in the toothing 48 of when a capacitive measuring field 52 is reached. The differences in the distance traveled at a defined pretensioning force can then be used as information about the skin type (soft, medium, hard) in order to determine the required depth t for puncturing and penetration. The contact member 16 as a probe is also in this connection ah integral component of the disposable 10.

The force can be detected before the lancing in a separate probing process or it can be part of the lancing process with a correspondingly more rapid evaluation of the force signal and drive control in real-time.

Embodiments of a lancing element 10 are shown in FIG. 12 and 13 with adjustable length stops which define a preset lancing depth of the lancing member 14 in the body part. These stops 54 are molded onto the lancing element 10 as integrated structures and provided with a stop face 56 located proximally behind the lancing member 14. As shown in FIG. 12a and 12b the stop position relative to the lancing element 14 is variable and executed by a bending deformation which is carried out in the instrument 18 before the lancing such that a lancing depth that is adapted to the body part can be permanently preset on the lancing element. This for example allows different skin types to be taken into account in order to reliably but at the same time painlessly reach the blood-yielding zone.

In the embodiment according to FIG. 13 a plurality of kinks 58 are preformed on the stops 54 in order to simplify the bending adaptation of the stop position. Alternatively a length adaptation can also be achieved by breaking off segments. The adjustment is made on a single part in the instrument 18 i.e. on the lancing element 10 so that it is not necessary to adjust several structural units. Variants are also conceivable comprising a lockable toothing similar to the reset lock described in connection with FIG. 6.

Thus, embodiments of the lancing element, lancing system and a method for skin detection are disclosed. One skilled in the art will appreciate that the teachings can be practiced with embodiments other, than those disclosed. The disclosed embodiments are presented for purposes of illustration and not limitation, and the invention is only limited by the claims that follow.

What is claimed is:

1. A lancing element for collecting a body fluid through skin, comprising:
a support;
a lancing member which rigidly projects from the support configured to pierce a skin in a lancing movement; and,
a pretensioning means coupled to the support which tightens the skin near a puncture site, the pretensioning means having a contact member that impacts the skin during the lancing movement and moves back during the lancing movement in an opposite direction to the lancing member.

2. The lancing element as in claim 1 where the contact member in an initial state is positioned in a direction of lancing to a side and in front of the lancing member or the contact member and the contact member is located at a same level or downstream of the lancing member and is reset against the support during the lancing movement while in contact with the skin.

3. The lancing element as in claim 2 wherein the contact member is supported on the support via a spring element.

4. The lancing element as in claim 2 wherein the contact member is locked in a reset position that is proximally set back relative to the lancing member by means of a locking mechanism.

5. The lancing element as in claim 2 wherein the contact member is moved together with the support when the support is retracted while reducing or removing a contact pressure on the skin.

6. The lancing element as in claim 1 wherein the pretensioning means has a coupling member that is elastically compressible and held by frictional lock that connects the contact member with the support.

7. The lancing element as in claim 1 wherein the coupling member is formed by a folding arm that is bent having at least one bending point.

8. The lancing element as in claim 1 wherein the pretensioning means is attached as an integral component of the support.

9. The lancing element as in claim 1 wherein the support, the lancing member, and the pretensioning means are formed uniformly from one material as a disposable part.

10. The lancing element as in claim 1 wherein the support, the lancing member, and pretensioning means are formed from a flat substrate that is etched and the pretensioning means is arranged as a part of the substrate in a substrate plane or is bent out of the substrate plane.

11. The lancing element as in claim 1 wherein the contact member has a point shaped or linear edge contour configured to be pressed against the skin laterally next to the lancing member.

12. The lancing element as in claim 1 wherein the lancing member is configured to be inserted into the skin at a lateral distance of less than 3 mm from the contact member.

13. The lancing element as in claim 1, further comprising,
a collecting structure that is capillary-active for collecting a body fluid.

14. The lancing element as in claim 1, further comprising,
a stop coupled to the support that limits a lancing depth of the lancing member, the stop having a skin contact surface that impacts at a defined proximal distance from the lancing member during the lancing movement.

15. The lancing element as in claim 14 wherein the stop length that projects from the base member is adjusted to adjust the lancing depth.

16. A lancing system for collecting a body fluid through the skin, comprising:
a support;
a lancing member which rigidly projects from the support configured to pierce skin in a lancing movement;
a pretensioning means coupled to the support which tightens the skin near a puncture site, the pretensioning means having a contact member that impacts the skin during the lancing movement and moves back during the lancing movement in an opposite direction to the lancing member; and,
a lancing drive for moving the lancing member forwards and backwards to create the lancing movement.

17. The lancing system as in claim 16, further comprising,
a position detector for detecting the position of the skin that has been tightened during the lancing movement of the lancing element.

18. The lancing system as in claim 16 wherein the lancing drive has a device for adjusting a lancing depth of the lancing member relative to a detected reference position of the skin that has been tightened.

19. The lancing system as in claim 16 wherein a distance measuring unit operates incrementally to detect a relative position of the contact member and a lancing member.

20. The lancing system as in claim 17 wherein the skin is supported on a circular receiver and the pretensioning means engages in the receiver.

21. The lancing system as in claim 17 wherein the position detector probes the skin that has been tightened by the pretensioning means using the contact member or the lancing member as a sensor to determine the skin position.

22. The lancing system as in claim 21 wherein the position detector detects a change in capacitance or conductivity or force when it probes the skin.

23. A system for skin detection during taking of body fluid samples, comprising:
a lancing element;
a position detector for detecting the position of a skin relative to a movement axis of the lancing element; and,
a pretensioning means for locally tightening the skin when the position detector detects the position of the skin relative to a movement axis of the lancing element.

24. The system as in claim 23 wherein the lancing element is moved into a retracted position from the skin after the position detector has detected the position of the skin and before the lancing element moves along the movement axis.

25. The system as in claim according to claim 23 wherein the pretensioning means is formed by a lancing member or a contact member of the lancing element.

26. The system as in claim 23 wherein the lancing element movement is controlled according to the detected position of the skin by a lancing drive in order to set a defined lancing depth.

27. A method for skin detection for taking samples of a body fluid, comprising:
detecting a position of a skin relative to a movement axis of a lancing element; and,
tightening locally the skin during detecting the position of the skin by a pretensioning means of the lancing element.

28. The method as in claim 27, further comprising,
moving the lancing element into a position that is retracted from the skin after detecting the position of the skin and before a lancing movement.

29. The method as in claim 27 wherein the pretensioning means comprises a lancing member or a contact member of the lancing element.

30. The method as in claim 28 wherein the lancing element is controlled according to a detected skin position in order to set a defined lancing depth.

31. A lancing element with skin pretensioner for lancing depth control while collecting body fluid through skin, comprising:
a support;
a lancing member coupled to the support and configured to rigidly project from the support to pierce skin during a lancing movement; and,
a pretensioner coupled to the support, the pretensioner having a contact member configured to impact skin during the lancing movement and move in a direction opposite to that of the lancing member during the lancing movement.

32. A lancing element with skin pretensioner for lancing depth control while collecting body fluid through skin, comprising:
a support;
a lancing member coupled to the support and configured to rigidly project from the support to pierce skin during a lancing movement; and,
means for pretensioning coupled to the support configured to impact skin during the lancing movement and move in a direction opposite to that of the lancing member during the lancing movement.

33. A method for controlling lancing depth during lancing to collect body fluid through a patient's skin, comprising:
detecting the position of a skin relative to movement of a lancing element;
tightening skin locally during detecting position of the skin with a pretensioner;
retracting the lancing element from the skin after detecting the position of the skin;
lancing the skin with the lancing element by advancing the lancing element into the skin;
controlling movement of the lancing element according to the position of the skin to set a defined lancing depth; and,
withdrawing the lancing element by moving the lancing element away from the skin.

34. The method as in claim 33, further comprising,
locking the lancing element at substantially a maximum advance of the lancing element so the pretensioner that has been compressed no longer presses against the skin while withdrawing the lancing element from the skin.
